(19) United States
(12) Patent Application Publication
(10) Pub. No.: US 2024/0268707 A1
(43) Pub. Date: Aug. 15, 2024

(54) INJURY PREVENTION SYSTEM FOR REMOTELY MONITORING SUBJECTS

(71) Applicant: Enhanced Surface Dynamics, Inc., Palo Alto, CA (US)

(72) Inventors: Amir Ben Shalom, Modiin (IL); Shlomo Hanassy, Ashdod (IL); Lior Greenstein, Tel-Aviv (IL); Tal Remez, Jerusalem (IL)

(21) Appl. No.: 18/644,822

(22) Filed: Apr. 24, 2024

Related U.S. Application Data
(63) Continuation of application No. 17/848,005, filed on Jun. 23, 2022, now abandoned.
Application No. 17/848,005 is a division of application No. 15/585,848, filed on May 3, 2017, now abandoned.
Application No. 15/585,848 is a continuation of application No. 14/372,030, filed on Jul. 14, 2014, now abandoned.
(60) Provisional application No. 61/586,405, filed on Jan. 13, 2012.
(60) Provisional application No. 61/586,408, filed on Jan. 13, 2012.
(60) Provisional application No. 61/593,988, filed on Feb. 2, 2012.
(60) Provisional application No. 61/593,992, filed on Feb. 2, 2012.
(60) Provisional application No. 61/684,369, filed on Aug. 17, 2012.

(51) Int. Cl. A61B 5/11; A61B 5/00; A61G 7/05; A61G 7/057; G01L 1/00; G01L 5/00; G06T 7/00; G16H 30/20; G16H 40/63; G16H 50/30

(52) U.S. Cl. CPC A61B 5/1115; A61B 5/1117; A61B 5/1128; A61B 5/447; A61B 5/6892; A61B 5/7275; A61B 5/7282; A61B 5/746; A61G 7/0527; A61G 7/057; G01L 1/00; G01L 5/00; G06T 7/0012; G16H 30/20; G16H 50/30; A61B 2562/0247; A61G 2203/34; A61G 2203/44; A61G 2203/70; G16H 40/63

(57) ABSTRACT

An injury prevention system includes a plurality of pressure sensing assemblies configured to measure pressure exerted upon a plurality of subjects over time. Each of the pressure sensing assemblies in the plurality of pressure sensing assemblies includes a hardware controller and a pressure sensor array. The injury prevention system includes a management system including a plurality of system control units and a remote unit. The remote unit is remote from the plurality of system control units. Each of the hardware controllers are configured to transfer output signals from each of the pressure sensor arrays to one of the system control units. The remote unit is configured to communicate with the plurality of system control units to monitor the plurality of subjects for injury prevention.

### CROSS-REFERENCE TO RELATED APPLICATIONS

This application is a Continuation of U.S. application Ser. No. 17/848,005 filed Jun. 23, 2022, which in turn, is a Divisional of U.S. application Ser. No. 15/585,848 filed May 3, 2017, which in turn, is a Continuation of Ser. No. 14/372,030 filed Jul. 14, 2014, now abandoned, which is a U.S. National Phase Application under 35 U.S.C. § 371 of International Application No. PCT/IB2013/050173, which has an international filing date of Jan. 9, 2013, and which claims the benefit of priority of U.S. Provisional Patent Application No. 61/586,405, filed Jan. 13, 2012, now expired, U.S. Provisional Patent Application No. 61/586,408, filed Jan. 13, 2012, now expired, U.S. Provisional Patent Application No. 61/593,988, filed Feb. 2, 2012, now expired, U.S. Provisional Patent Application No. 61/593,992, now expired, filed Feb. 2, 2012, and U.S. Provisional Patent Application No. 61/684,369, filed Aug. 17, 2012, now expired, the disclosures of which are hereby incorporated in their entirety by reference herein.

### TECHNICAL FIELD

The disclosure herein, in some embodiments thereof, relates to systems and methods for remotely preventing injuries (e.g., pressure ulcers).

### BACKGROUND

Pressure wounds e.g. decubitus ulcers, which are commonly known as pressure ulcers or bedsores, are lesions developed when a localized area of soft tissue is compressed between a bony prominence and an external surface for a prolonged period of time. Pressure ulcers may appear in various parts of the body, and their development is affected by a combination of factors such as unrelieved pressure, friction, shearing forces, humidity and temperature.

Currently, about 10%-15% of hospitalized patients are estimated to have bedsores at any one time (Source: Medicare website 2009). However, it is not only hospitalized patients who suffer from pressure wounds: for example, people confined to wheelchairs are prone to suffer from pressure wounds, especially in their pelvis, lower back and ankles. Although easily prevented and completely treatable if found early, bedsores are painful, and treatment is both difficult and expensive. In many cases bedsores can prove fatal—even under the auspices of medical care.

The most effective way of dealing with pressure wounds is to prevent them. Existing preventive solutions are either passive (e.g. various types of cushioning) or active, including a range of dynamic mattresses that alternate the inflation/deflation of air cells. Typically, such mattresses re-distribute pressure even from unnecessary locations thereby needlessly creating higher pressure in sensitive areas. Moreover, such mattresses are typically designed for patients lying down in hospital beds, and hardly answer the needs of individuals who spend considerable amounts of time sitting up, confined to a wheelchair or the like.

The most common preventive approach is keeping a strict routine of relieving pressure from sensitive body areas of a patient every 2-3 hours. This can be done with patients under strict medical care. Apart from being a difficult, labor intensive and costly task, it does not meet the needs of independent individuals who do not require ongoing supervision of a caretaker, such as paraplegics who use a wheelchair for mobility.

### SUMMARY

In a first embodiment, an injury prevention system is disclosed. The injury prevention system includes a plurality of pressure sensing assemblies configured to measure pressure exerted upon a plurality of subjects over time. Each of the pressure sensing assemblies in the plurality of pressure sensing assemblies includes a hardware controller and a pressure sensor array. The injury prevention system includes a management system including a plurality of system control units and a remote unit. The remote unit is remote from the plurality of system control units. Each of the hardware controllers are configured to transfer output signals from each of the pressure sensor arrays to one of the system control units. The remote unit is configured to communicate with the plurality of system control units to monitor the plurality of subjects for injury prevention.

In a second embodiment, a pressure sensing system is disclosed. The pressure sensing system includes a pressure sensing assembly comprising pressure sensing elements situated at known points represented by a location vector in a first coordinate system of the pressure sensing assembly. The pressure sensing system further includes a non-transitory computer-readable medium having computer-readable instructions stored thereon that are configured to be executed by a processor to perform the following functions. The functions include recording pressure values representing pressure exerted by a subject on the pressure sensing elements, associating the recorded pressure values with the location vector of the coordinate system, and transforming the associated location vector into a mapped vector in a second coordinate system to obtain a pressure distribution image.

### BRIEF DESCRIPTION OF THE DRAWINGS

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1A is a block diagram showing the main elements of an injury prevention system incorporating an example of a management device and a pressure sensing assembly;

FIG. 1B is a block diagram showing the main elements of an example of the injury prevention system comprising a modular pressure wound prevention system;

FIGS. 2A and 2B are a top view and section through, respectively, of a further example of a sensor module incorporated into a mattress overlay;

FIG. 3 is a flowchart of a method for using a modular pressure wound prevention system;

FIG. 4A is a flowchart showing a possible method for registering a bed exit event;

FIG. 4B is a flowchart showing another possible method for registering a bed exit event, using a total weight exerted calculation;

FIG. 4C is a flowchart showing another possible method for registering a bed exit event, using a relative weight exerted calculation;

FIG. 5A is a flowchart showing a possible method for registering a bed fall risk event, using an optional subject mapping analysis;

FIG. 5B is a flowchart showing a possible method for registering the bed fall risk event, using an overlapping analysis;

FIG. 5C is a flowchart showing a possible method for registering the bed fall risk event, using a posture identification analysis;

FIG. 6 shows a possible layout with margin definition used for calculation of the bed fall risk event;

FIG. 7 shows a possible screen shot monitoring an occurrence of a bed fall event.

FIG. 8 is a flowchart showing a possible method for presenting pressure data related to the risk of a subject developing pressure injuries;

FIG. 9 is a flowchart of another method for determining the risk of a subject developing a pressure injury;

FIGS. 10A-F show a selection of some common postures, which, amongst others, may adopted by subjects recumbent upon a horizontal surface;

FIG. 11 is a flowchart representing selected actions of a method for identifying a recumbent posture;

FIG. 12 is a flowchart representing selected actions of a method for identifying a sequence of postures sequentially adopted by a subject;

FIG. 13 shows a possible screenshot for displaying a sequence of postures to a caregiver;

FIGS. 14A-D show four pressure distribution maps and associated pressure histograms recorded by a pressure sensing assembly for a subject adopting various recumbent postures;

FIGS. 15A and 15B are graphical illustrations of a coordinate system of the pressure sensing assembly and a coordinate system of a subject body respectively;

FIGS. 16A and 16B represent a possible pressure distribution image and an associated body model, respectively, showing pressure distribution for a subject in a supine posture;

FIGS. 17A and 17B represent a possible pressure distribution image and an associated body model, respectively, showing pressure distribution for a subject in a prostrate posture;

FIG. 18 is a flowchart representing a method for mapping a coordinate system of a pressure sensing array to a body coordinate system;

FIG. 19 is a flowchart representing selected actions of a method for identifying a recumbent posture;

FIGS. 20A-C are a selection of flowcharts of methods for analyzing and reducing possible noise patterns; and

FIG. 21 is a flowchart representing selected actions of a method for identifying subject movements.

### DETAILED DESCRIPTION

The disclosure herein, in some embodiments thereof, relates to systems and methods for prevention of injury and, more particularly, but not exclusively, to systems and methods for managing risk of a subject developing an injury. The system may use sensing apparatus to determine the indications that a subject may be developing a pressure injury, or may be otherwise at risk, for example of falling from a bed or the like. According to some aspects of the current disclosure, additional methods for risk indication management functional analysis are disclosed. In particular a risk index function is presented as well as the identification of recumbent postures adopted by a subject and their use in determining risk of developing pressure injuries.

It is also noted, particularly, that the disclosure is related to mapping coordinates of pressure sensing elements of a pressure sensing assembly or mat, to a subject-based coordinate system.

It is further noted that, the disclosure relates to methods for improving the monitoring of the pressure image indicating a patient's pressure distribution used in several algorithms of the system.

It is noted that the systems and methods of the disclosure herein may not be limited in its application to the details of construction and the arrangement of the components or methods set forth in the description or illustrated in the drawings and examples. The systems and methods of the disclosure may be capable of other embodiments or of being practiced or carried out in various ways.

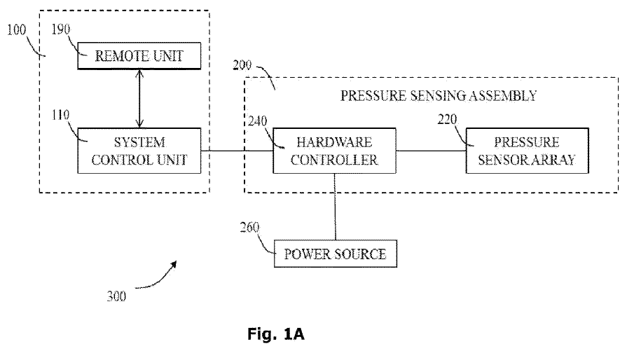

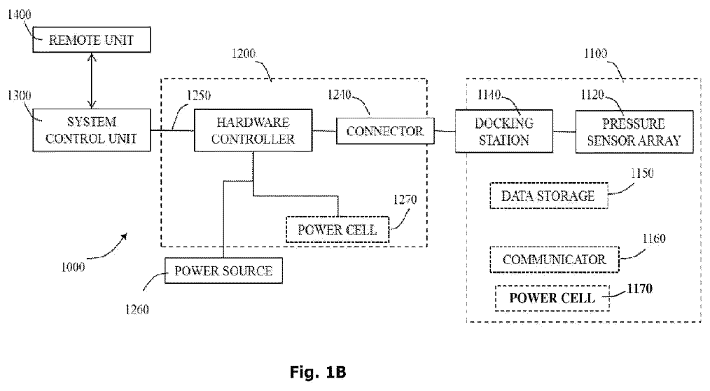

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting. Reference is now made to the block diagram of FIG. 1A showing selected elements of an injury prevention system 300 incorporating an example of a management system and a pressure sensing assembly. The injury prevention system 300, such as a pressure wound prevention system for example, may include a pressure sensor assembly 200 and a management system 100 and may be used to prevent the development of pressure related injuries such as decubitus ulcers or the like. Examples of such a pressure sensor assembly are described in copending applications PCT/IL2010/000294, PCT/IB2011/051016 PCT/IB2011/054773, PCT/IB2012/050829 and PCT/IB2012/053538, all of which are incorporated herein by reference.

The pressure sensor assembly 200 is provided to measure the pressure exerted upon a subject over time. The pressure sensor assembly 200 includes a pressure sensor array 220, a hardware controller 240 and optionally a data storage device unit (not shown). The hardware controller 240 may be configured to provide power and analog or digital control to the pressure sensor array 220. The hardware controller 240 may be further configured to transfer output signals from the pressure sensor assembly 200 to the management system 100.

The pressure sensor array 220 may be a pressure sensing mat configured to be placed between a mattress and the body of a bed-bound patient, for example. Other examples of the pressure sensor array 220 may include pressure sensitive pads, cushions, clothing or the like.

The management system 100 includes a system control unit 110, provided to control the settings and operation of the pressure sensing assembly 200 as well as to provide output from the system to a user. The management system 100 may warn and/or alert a caregiver to potential risk of the subject developing pressure wounds. Optionally the system control unit 110 is in communication with a remote unit 190 enabling a user to configure settings and monitor the output of the system remotely. It will be appreciated that a remote unit 190 may be further configured to communicate the output from a plurality of system control units 110 such that the remote user may be able to monitor a plurality of subjects. This may be of particular use, for example, in a hospital setting, where a bedside system control unit 110 may be configured to communicate with a remote unit 190 at a nurse's station for example. The remote unit 190 may be further configured to record data in a data storage device unit for subsequent retrieval.

Reference is now made to the block diagram of FIG. 1B showing an example of a modular system 1000 which may be used to prevent pressure wound formation. The system 1000 includes a sensor module 1100 and two management modules: a hardware controller module 1200 and a system control module 1300. Optionally the system 1000 may further include a remote unit 1400 in communication with the system control unit 1300 and via which the system 1000 may be controlled and its output monitored.

The pressure wound prevention system 1000 may be used to monitor pressure distribution between a subject and a surface and to warn and/or alert a caregiver to potential risk of the subject developing pressure wounds. Using such a pressure wound prevention system may therefore enable the caregiver to take preventative action such as turning or otherwise repositioning the subject before pressure wounds develop.

It is a particular feature of the modular system 1000 that the various modules may be exchanged or replaced independently from one another. This may be an important aspect of systems in which different modules have different lifespans and a shorter lifespan module may be replaced without exchanging modules with longer lifespans.

The sensor module 1100 includes a pressure sensor array 1120 and a docking station 1140. The array of pressure sensors 1120 is configured to measure pressure between the subject and a surface. The docking station 1140 may provide a communication channel between the sensor array and the management modules 1200, 1300.

The sensor module 1100 may further include a number of additional elements such as a data storage unit 1150, communicator 1160 or integrated power cell 1170, for example. The data storage unit 1150 may be configured to store historical data relating to the sensor module 1100 for use in calculation of risk factors or display history of events flow. For example, such data may include records of initial values of certain reference parameters as well as their variation with time so as to provide corrections to pressure measurements.

It will be appreciated that the inclusion of a data storage unit 1150 such as a memory chip or the like may enable the sensor module 1100 to be readily connected to different management modules with a continuity of record being maintained. Thus, for example, in a hospital environment a bed-bound patient is often moved from one ward to another while remaining in the same bed. The sensor module 1100, such as the overlay sheet described herein below, may be disconnected from a first management module in the first ward and reconnected to add a new management module in a new ward with no loss of continuity to the data record.

A communicator unit 1160 may be provided to communicate data to the management modules. Where appropriate, wireless communicators may provide a communication channel via transceivers such as radio transceivers, inductive transfer systems or the like, possibly using known protocols such as WiFi, Bluetooth, NFC or the like.

The hardware controller module 1200 is configured to receive analog sensor signals from the sensor module 1100 via a connector 1240 coupled to the docking station 1140. Analog sensor signals received from the sensor module 1100 may be transferred to the system control unit 1300. Additionally, the hardware controller module 1200 may provide power to the sensor module 1100, possibly from an external power source 1260 or an internal power cell 1270. Alternatively, the sensor module 1100 may comprise its own on board power cell (not shown) such as a battery pack or the like.

The system control unit 1300 is provided to control the settings and operation of the system 1000 as well as to provide output from the system to a user. The system control unit 1300 may be connected to the hardware controller 1200 via a communication line 1250. It is noted that the communication line may be a wired communication cable, a wireless link or such like. It is particularly noted that in hospital settings, where equipment such as beds and the like are often moved around, a robust communication cable is desirable, accordingly an extendable, coiled, helical or other cable may be used such that it tends to extend rather than to break when snagged.

Optionally the system control unit 1300 is in communication with a remote unit 1400 enabling a user to configure settings and monitor the output of the system 1000 remotely.

The docking station 1140 is configured to couple with a connector 1240 of the hardware controller 1200 and may provide a communication channel between the sensor array 1120 and the management modules 1200, 1300. In addition the docking station 1140 may provide a power line to connect the sensor array 1120 to a power source 1260.

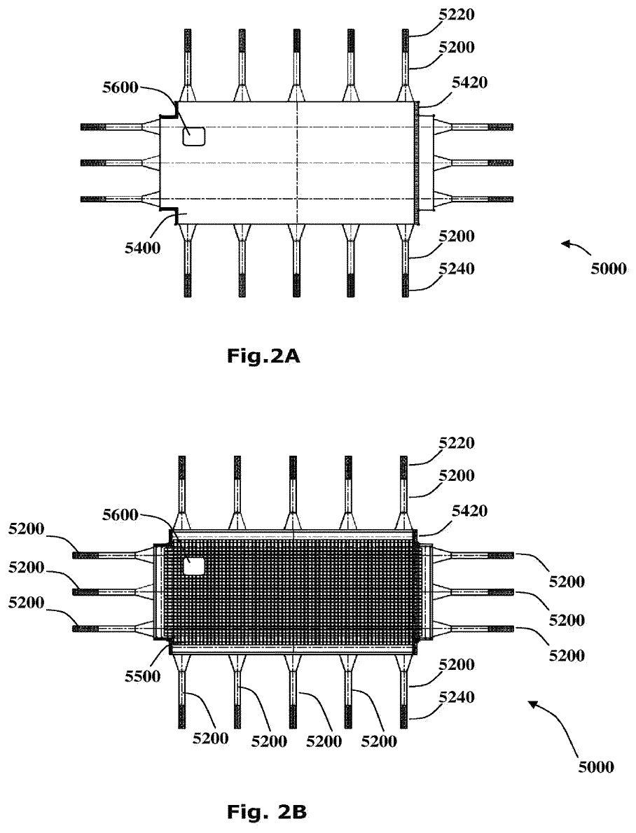

With reference to FIGS. 2A and 2B, a top view and section through are shown respectively of an example of a sensor module incorporated into a mattress overlay 5000. The overlay 5000 incorporates a sensor matrix 5500 and docking station 5600, such as described hereinabove. The sensor matrix 5500 is housed within a cover sheet 5400 and which may be sealed by a zipper 5420 or alternatively sewn into the cover as required. The sensor module may be connected to a hardware controller (not shown) via the docking station 5600.

The pressure detection mat 5000 may be attached to a surface in such a way that prevents movement of the mat relative to the surface. A feature of the embodiment of the mat 5000 is that the cover sheet 5400 may include a coupling mechanism for securing the mat to a seat or a back of a mattress, a bed, a chair, a bench, a sofa, a wheelchair or the like. The coupling mechanism may include for example at least one strap 5200 having an attachment means 5240 configured to secure the straps 5200 to the seat or to each other such that the pressure detection mat is held securely. This may be useful to prevent folding, wrinkling or other movement of the detection mat which may contribute to the creation of shear forces which are known to encourage the formation of external pressure sores. Suitable attachment means include for example, hook-and-eye materials such as Velcro®, buckles, adhesives, buttons, laces or the like as suits requirements.

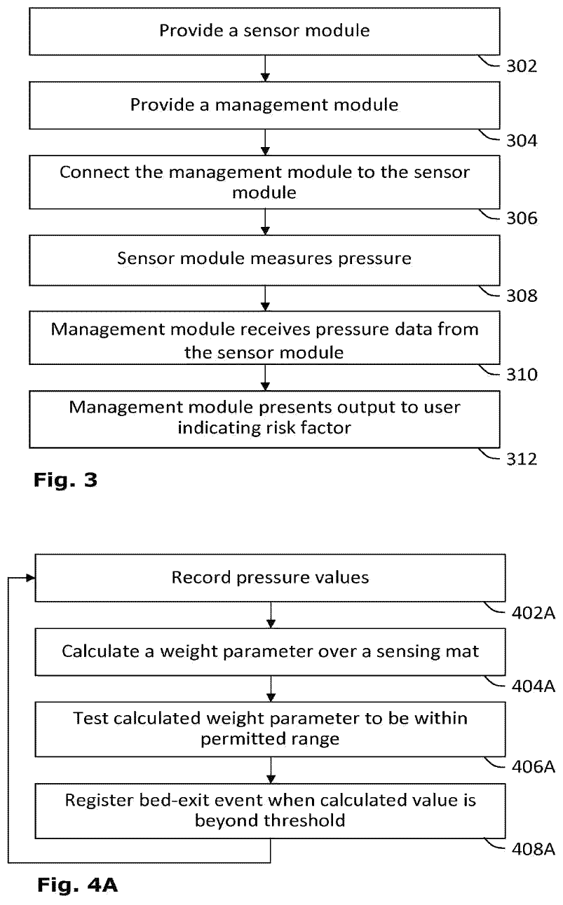

Referring to the flowchart of FIG. 3 a method is disclosed for the prevention the development of pressure wounds. The method includes the steps: providing a sensor module 302; providing a management module 304; connecting the management module to the sensor module 306; the sensor module measuring pressure exerted by the subject 308, the management module receiving pressure data from the sensor module 310; and the management module presenting an output to a user indicating risk of subject developing pressure wounds 312.

A processor of the system control unit 1300 may be configured to receive settings via a user input apparatus and to monitor data via a data input. The processor may be further configured to measure elapsed time of monitoring, typically using an internal clock. The processor is operable to calculate a risk index, based upon settings and monitored data, which relates to the corresponding chance that a monitored subject will develop injuries such as pressure wounds. By comparing the calculated risk index with certain threshold values, the processor may be operable to alert a user to necessary actions required to adequately care for a subject.

It will be appreciated that there are a plurality of factors influencing the probability that pressure wounds may develop in a subject. Monitored pressure alone may be a limited indicator of risk of developing pressure wounds because pressure wounds develop as a result of pressure being sustained over a prolonged duration. Accordingly, in certain systems, the elapsed time Δt, alone, may be used as one risk factor R₁. In other systems, an additional risk factor R₂ may be calculated from the product of pressure exerted P with the time Δt during which the pressure was recorded, such that the risk factor is given by the formula R₂=PΔt.

Optionally, in selected embodiments, a visual display unit may be configured to display a map of risk indices instead of or as well as a pressure map. The risk index map may be color coded to allow a user to readily identify the areas which are at the highest risk of injury.

The displayed map may represent a two dimensional representation of a pressure sensing map with each pixel being colored according to a corresponding pressure sensing element. A caregiver may be able to identify thereby which areas of the subject's body correspond to the areas on the map display.

Alternatively or additionally, where possible the display may be configured to map each pixel to a point upon the body of the subject thereby indicating the risk index of each region directly on the body of a subject. It will be appreciated that such a mapping may allow the continued monitoring of the same areas of a subject even when the subject has been repositioned.

Furthermore, the threshold risk index may not be uniform for the whole body of a subject. Therefore, certain systems may be configurable such that different sets of 30 pressure sensors have different risk index thresholds. Where appropriate, threshold values for each set of pressure sensors of a pressure sensing apparatus may be set independently by a user. It will be appreciated, however, that where possible, threshold values may be set according to body areas.

Thus in particular systems, the processors may be further configured to perform pattern recognition of the monitored subject so as to identify the various body regions and to calculate risk indices and set threshold values accordingly. This is particularly useful, for example where a sensor mat may become shifted beneath the body of a subject, without the subject's body being significantly repositioned.

More generally, however, various risk factors may be considered in the generation of a risk index, such as pressure exerted, time elapsed and sensitivity of the body tissue upon which the pressure is exerted.

The pressure risk index p(P(τ,x,y)) may be considered as a function of pressure P measured at a given point (x, y) and at a given time t.

The sensitivity of the body tissue sᵤ(x,y,w,a) at a given point (x, y) may depend upon a range of medical influences, as discussed below, in particular the sensitivity may depend upon the subjects weight w and age a.

It is further noted that the pressure effect is typically time additive and this may be reflected in a calculation of overall risk index function r(τ,x,y) for example as:

$$r ( τ , x , y ) = {\sum }_{t = 1}^{τ} K ( t - τ ) * p ( P ( τ , x , y ) ) * {s}_{u} ( x , y , w , α )$$

where K(t−τ) is a time kernel function representing the additive effects of pressure.

The above described functions may be determined by experimental data. Such experimental data may be harvested, at least in part, from existing published literature. The frequency of the development of stress ulcers and the like at various areas of the body may be counted for subjects of various ages, weights, and genders as well as for different medical conditions, all of which may be included in more detailed risk factor functions. Thus a probabilistic model may be generated. Furthermore, additional data may be gathered from ongoing monitoring of subjects using pressure sensing systems. It will be appreciated that, over time, as the data reservoir grows, the accuracy of the risk factor functions may be improved.

The monitored pressure is generally measured by a pressure sensor array 220 of a pressure sensor assembly 200 (FIG. 1A). Accordingly, each pixel of the pressure sensor array 220 may correspond to a point (x, y) upon a two dimensional surface over which pressure P is measured. The sensitivity of the body tissue sᵤ(x,y,w,a) depends upon the point upon the surface of the body upon which pressure is exerted.

Where the subject is largely stationary, it may be possible to use the coordinates of the mattress to calculate the risk index for each point thereof. Accordingly, the risk index may need to be recalculated when the subject is repositioned such that a different body part comes into contact with the monitored pixel. However, where possible, it may be useful to map the coordinate system of the two dimensional surface over which pressure P is measured to the surface of the body.

In order to map the pixel measurements to the body coordinate system various techniques may be used to identify body posture of the patient or to otherwise recognize body features. Many algorithms are known in the art which may be used towards this end, such as particle component analysis, support vector machine, Kmean, two-dimensional fast Fourier analysis, earth movers distance and the like. In particular the earth mover distance (EMD) algorithm is a method to compare between two distributions, and which is commonly used in pattern recognition of visual signatures. The EMD algorithm may be readily applied, for example, to compare between a recorded posture and candidate posture types stored in a database.

The output mechanism may be configured to respond to changes in monitored risk index and to alert a user appropriately. For example if a risk index exceeds a threshold value, the alarm may be sounded alerting a caregiver to the immediate necessity to reposition the subject. Similarly, when the risk index approaches such a threshold an alert may be displayed alerting a caregiver to the imminent need to reposition the subject or the like. Alternatively, different risk factors may trigger different alerts when they reach their threshold values.

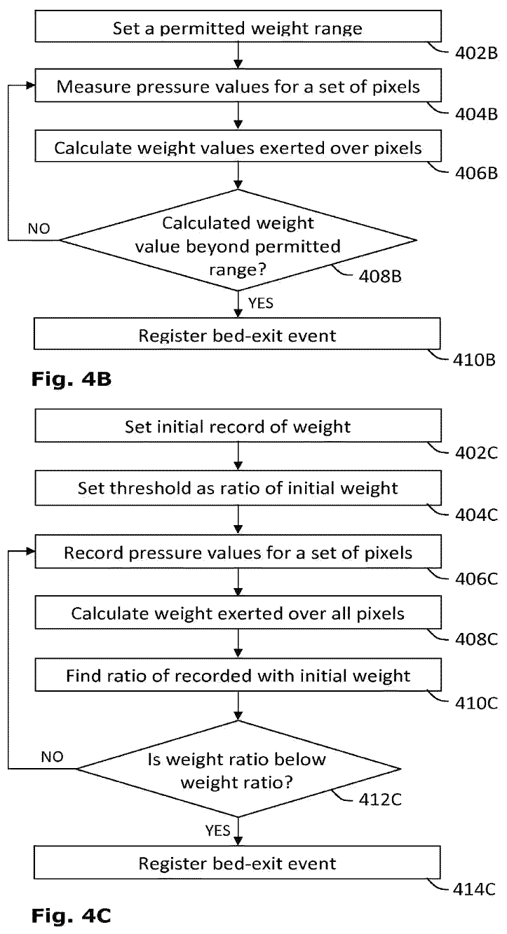

Referring now to the flowchart of FIG. 4A selected actions are indicated of a method for determining the occurrence of a bed becoming unoccupied. The method may be used in an injury prevention system.

Detection of bed occupancy may be a functionality feature of an injury prevention system. A bed exit event may indicate that a bed changed from a state of being occupied to a state of being unoccupied; in other words the subject has left the bed, intentionally or unintentionally. Where a subject leaving a bed may require attention of a caregiver, a bed exit event may warrant a response functionality of an injury prevention system. The injury prevention system may be configured, for 5 example, to warn or alert when such an event is registered in order to enable a pause or cessation of monitoring or to initiate further actions that may be required automatically or manually.

The method may use recorded pressure values from a plurality of pressure sensing elements to calculate the total weight exerted over a surface, using a pressure sensitive mat, for example. The calculated weight value may be compared with a permitted weight range to determine occurrence of such an event. Alternatively or additionally, weight may be measured directly using a scale. Optionally, weight may be input manually by a caregiver, by the subjects themselves or the like.

According to the method, pressure may be measured by a plurality of pressure sensing elements—step 402A. Such data may be recorded using a pressure sensing mat such as described hereinabove, for example. A weight parameter may be calculated based on the recorded pressure values—step 404A. The weight parameter may be, for example, the total weight exerted by a subject over a sensing mat, a relative weight value, or some other parameter indicative of weight. The calculated weight parameter may be tested to determine whether or not it is within a permitted range—step 406A. The calculated weight parameter being beyond a threshold value indicates the occurrence of a changing of state from occupied to unoccupied, and thus, a bed exit event may be registered—step 408A. Following the registration of a bed exit event, the method may stop. Otherwise, the cycle may be repeated. Optionally, upon registration of a bed exit event, a warning and/or alert may be signaled as part of the operation of the injury prevention system.

The method to determine change of bed state from occupied to unoccupied involves calculations that may be performed by a simple comparison to a predetermined weight range, to monitor pressure and calculate weight exerted as described in FIG. 4B hereinafter. Optionally, other calculation functions may be used, such as those based upon relative weight comparisons with an initially recorded weight, as described in FIG. 4C hereinafter. Alternatively, both functions of calculations may be used, simultaneously or sequentially, in any order of activation.

Referring to the flowchart of FIG. 4B, a calculation method is disclosed to register a bed exit event of a bed becoming unoccupied, by monitoring the weight exerted by the subject over a surface, a sensing mat for example.

The method may include setting a permitted weight range—step 402B. The permitted weight range may optionally be defined as a predetermined default value fetched from a data storage device or entered by user input. Alternatively, the permitted weight range may be provided as part of the calculation flow.

The weight range may be defined, for example, by a minimum threshold. A monitored weight below this minimum threshold may indicate a bed exit event, i.e., that the bed is not occupied.

Pressure values may be measured by a plurality of pressure sensing elements defining a set of pixels, and recorded—step 404B. The measuring and recording of pressure values may proceed in an ongoing manner, as a continuous process.

A weight value over the pixels may be determined from the pressure measurements—step 406B. The determination may be done, for example, by summing the pressure values for each pixel to get the total pressure exerted over the sensing mat and multiplying this value by the total pixel area to find the total weight of the subject exerted over all pixels of the sensing mat.

The calculate weight value, as determined in 406B, may be compared with the permitted range defined in step 402B (408B). If total weight is outside the permitted range, a bed exit event may be registered—step 410B. Optionally, upon registering of a bed exit event a signal may be triggered as described hereinabove, for example, to initiate an action such as an alert or the like.

Referring to the flowchart of FIG. 4C, a further method is disclosed to register a bed exit event of a bed becoming unoccupied, by monitoring the relative change in the weight applied over a surface, e.g., a sensing mat. This method may require an additional calibration phase, as defined hereinafter.

The method may include the calibration steps of setting the initial weight record, step 402C and setting a minimum threshold weight ratio value as a defined ratio compared against the initial weight record—step 404C. The initial weight record may be determined from an actual weight value as measured, when the subject is on a surface, such as a sensing mat. Additionally or alternatively the initial weight may be given a default value. In some systems, the default value may be read from a data storage device or entered by user input. Similarly, the minimum threshold weight ratio value may be given a default value, be fetched from a data storage device or read from a value entered by user input.

Pressure values may be measured by a plurality of pressure sensing elements defining a set of pixels, and recorded—step 406C. The measuring and recording of pressure values may proceed in an ongoing manner, as a continuous process.

A weight value over the pixels may be determined from the pressure measurements—step 408C. The determination may be done, for example, by summing the pressure values for each pixel to get the total pressure exerted over the sensing mat and multiplying this value by the total pixel area to find the total weight of the subject exerted over all pixels of the sensing mat.

The calculate weight value may be divided with the initial weight record as set in the calibration step 402C, thus defining an actual weight ratio—step 410C.

The actual weight ratio may be compared to the threshold weight ratio as set in step 404C—step 412C.

If the actual weight ratio is below the threshold weight ratio, then a bed exit event may be registered—step 414C. Optionally, upon registration of a bed exit event, an alert signal or the like may be triggered as described herein.

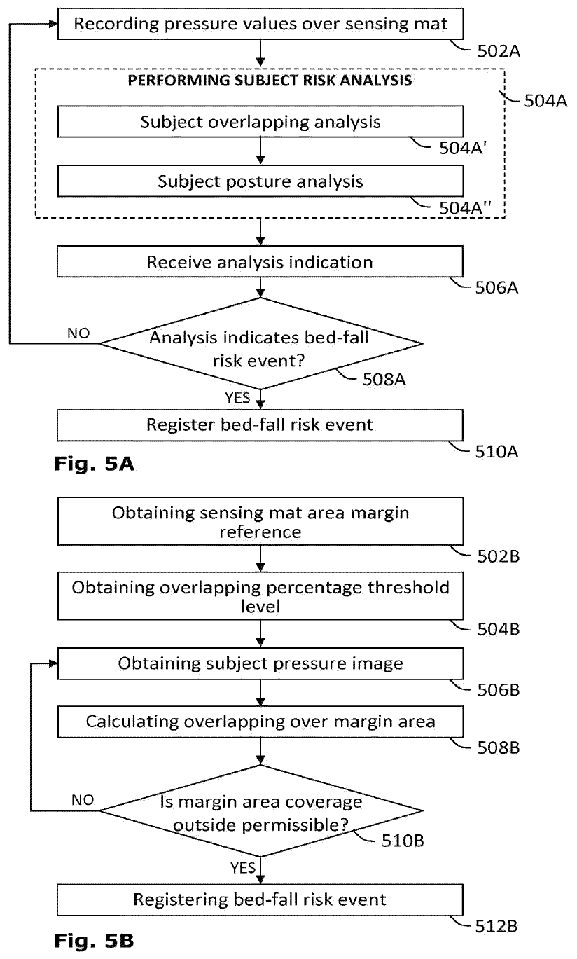

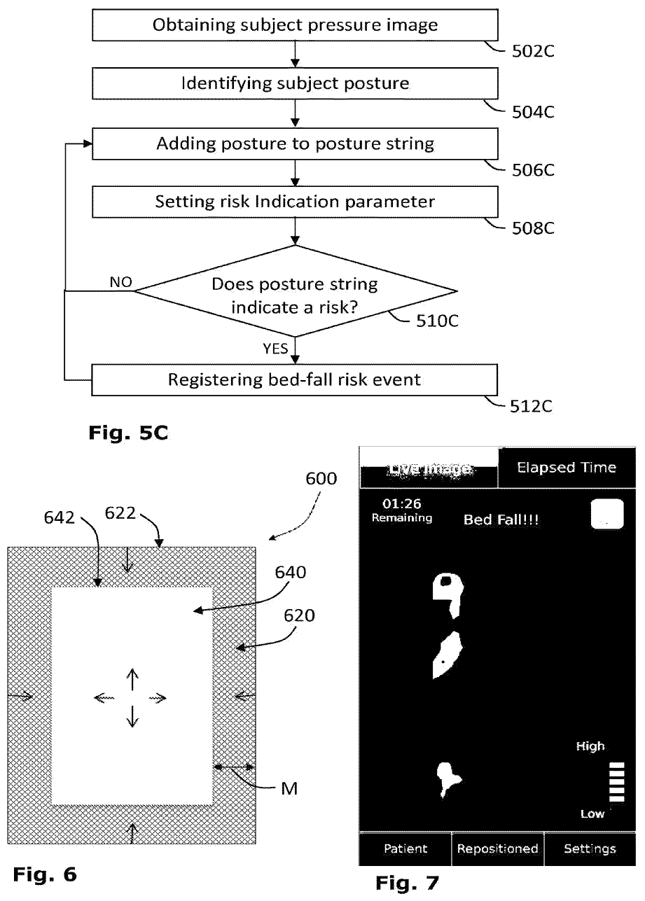

Referring now to the flow chart of FIG. 5A, selected actions are presented of a method for determining the risk of a subject falling from a surface, possibly accidentally, for example by rolling off a bed, slipping from the bed or the like. Such a risk is referred to herein as a bed fall risk. A method for determining bed fall risk may be useful in an injury prevention system.

Detection of rolling off a bed or the warning of the risk for such a situation may be a functionality feature of an injury prevention system. The occurrence of a high risk of imminent bed fall may be termed a bed fall risk event and may be used to trigger an automatic response, such as an alarm, the provision of a cushion or barrier or the like to prevent a fall or minimize harm caused thereby.

It will be appreciated that a bed fall risk event may precede a bed exit event indicating a bed state transitioning from occupied to unoccupied. The combination of a bed fall risk event and a bed exit event may indicate that a subject has fallen unintentionally from a bed. Alternatively, the bed exit event and the bed fall risk event may be unrelated and require different settings in resolving relevant post event responses, optionally by triggering different functionality of a medical monitoring process provided by an injury prevention system, by triggering an automatic response mechanism, by alerting a caregiver or the like.

The method may include the use of recorded pressure values from a plurality of pressure sensing elements to perform risk analysis for a subject recumbent upon a monitoring surface, for example, a pressure sensing mat. It is particularly noted that a pressure sensing mat operable to monitor pressure distribution over the surface may be particularly useful in determining the bed fall risk. The method may register any relevant bed fall risk event as described hereinabove, reflecting the result of the above risk analysis.

Pressure or pressure distribution may be measured by a plurality of pressure sensing elements to create a pressure image of a subject over a surface—step 502A. Such data may be recorded using a pressure sensing mat such as described hereinabove, for example. The pressure image may form the basic input for performing a subject risk analysis—step 504A. The subject risk analysis of a subject may include analyzing subject overlapping 504A′, analyzing posture indication 504A″ and/or some other method for determining risk. The risk analysis may indicate the risk of a subject rolling off or slipping from the bed and may be used to detect the bed fall risk event. The execution of these actions may be carried out simultaneously or sequentially, in any order of activation.

The subject risk analysis indication may be received (step 506A), and tested to determine whether or not to register a bed-fall risk event—step 508A.

If analysis indicates a bed-fall risk event, the bed-fall risk event is then registered—step 510A. If a bed-fall risk event is registered, the cyclic flow may be stopped. Otherwise, if no bed-fall risk event is registered, the hereinabove flow may continue repeatedly.

Optionally, upon registration of a bed fall risk event, a response signal may be triggered, for example, to provide a warning and/or alert, to initiate an automatic harm prevention response or the like as part of the operation of an injury prevention system. In addition, the registration of a bed fall risk event may pause or stop monitoring and the like, for example.

Referring to the flowchart of FIG. 5B, a calculation method is disclosed for registering a bed fall risk event, using a predefined margin area of a surface area, e.g., a margin of a sensing mat.

The method may include obtaining a sensing mat area margin reference defining an area within which subject overlap will be determined—step 502B. An overlapping percentage threshold level may be obtained to define the threshold beyond which the bed fall risk event may be registered—step 504B.

The values obtained in step 502B and step 504B may be defined as default values internal to the calculation flow. Alternatively, the values may be fetched from a data storage device or may be entered by user input.

The method may include the use of recorded pressure values from a plurality of pressure sensing elements to build a subject pressure distribution image over a surface, a sensing mat for example—step 506B. The pressure distribution image changes according to movement of a subject over the surface and may be obtained repeatedly to calculate subject positioning.

Accordingly, the overlapping area of a subject over the margin area may be calculated—step 508. The calculated margin area coverage is compared with a permitted range, for example against a threshold overlapping percentage level or the like—step 510B. A risk indication parameter may be thereby determined and a bed-fall risk event may be registered optionally pending risk indication analysis—step 512B.

Optionally, upon registration of bed-fall risk event, a response signal may be triggered as described hereinabove. Additionally, the cyclic flow may continue or alternatively may be stopped. If no registration of any bed fall risk event takes place, the hereinabove flow continues repeatedly.

Referring to the flowchart of FIG. 5C, a further calculation method is disclosed to register a bed fall risk event using a value of risk identification parameter determined from a posture analysis.

The method may include use of recorded pressure values from a plurality of pressure sensing elements to build the subject pressure image over a surface, a sensing mat for example—step 502C. It will be appreciated that the pressure image may change as the subject moves and adopts different postures over the surface and may therefore be obtained repeatedly.

Accordingly, subject posture based on a current pressure image may be identified—step 504C. The current posture may be added to an accumulative posture string—step 506C. Such a posture string may enable buildup of the risk identification parameter. The posture string may be tested against reference strings to determine if a risk is indicated—step 508C. Where a risk is indicated, the system may be operable to register a bed-fall risk event.

Optionally, upon registration of bed fall risk event a signal may be triggered as described hereinabove. Additionally, the cyclic flow may continue or alternatively may be stopped. If no registration of any bed fall risk event takes place, the hereinabove flow continues repeatedly.

The layout of FIG. 6 shows a possible margin definition 600 dividing the surface over which the subject is lying such as a mattress, for example into two zones: a high risk zone 620 and a low risk zone 640. The outer rectangular border 622 encloses the surface over which the subject is lying, and the inner rectangle 642 divides the surface into the low risk zone 640 and the high risk zone 620. The low risk zone 640 may be defined by selecting an inner border 642 within the outer rectangular frame lines 622 of the surface. Where the limit of the outer border 622 is known, the inner border 642 may be selected by reducing the outer border 622 by a margin value M. Alternatively, or additionally, the inner border 642 may be constructed by measuring a given distance in each direction from the center of the surface. Thus, the margin definition may be measured from the contour of the external rectangular surface, inside, towards its center or may be defined as a value measured from its center towards the outer frame.

The term low risk as used herein may refer to a calculated risk of a subject to fall from a surface to be below an acceptable limit. Accordingly, a high percentage of a subject's pressure image lying over the low risk zone 640 within the boundaries of the inner rectangular frame 642 may indicate a low risk of bed-fall. Conversely a high percentage of a subject's pressure image lying over the high risk zone 620 outside the boundaries of the inner rectangular frame 642 may indicate a high risk of bed-fall and an alert may be provided as required.

With reference now to FIG. 7 a possible screenshot is shown of a user interface, monitoring an occurrence of a bed fall event. This screen shot indicates a text message notifying the situation of a bed fall event and showing a subject's pressure image lying over the high risk zone of the surface.

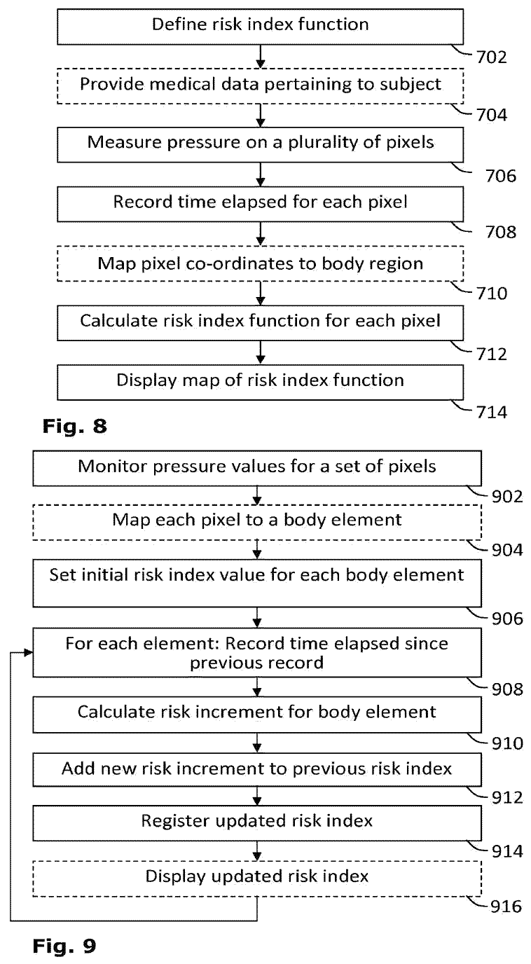

Referring now to the flowchart of FIG. 8, the main steps of a method are presented for determining and displaying pressure related measurements for use in an injury prevention system. The method uses recorded pressure values from a plurality of pressure sensing elements to generate useful values of risk index and to indicate these on a map.

The method may include defining a risk index function—step 702. The risk index function may be a function such as the accumulated pressure risk factor R₂=PΔt, based on the product of pressure exerted P with the time Δt during which the pressure was recorded, as described hereinabove. Alternatively, the risk index function may consider other relevant factors such as tissue type, condition of patient, region of the body and the like. Accordingly, relevant medical data pertaining to the subject may be provided to the system—step 704.

The pressure may be measured by a plurality of pressure sensing elements—step 706. Such data may be recording using a pressure sensing mat such as described hereinabove, for example. Other pressure sensing means may be alternatively used. The time elapsed during which pressure is measured for each pressure sensing element may be recorded—step 708.

Optionally, the pixel coordinates may be mapped onto a two dimensional array, to the plurality of pressure sensing elements. Alternatively, the pixel coordinates may be mapped to a body-based coordinate system—step 710. The body based coordinate system may allow the risk index to be calculated for each region of the body, which may be relevance to some defined risk index functions as described hereinabove.

A value for the risk index function may be calculated for each pixel—step 712. It is noted that values may be calculated for each pressure sensing element, based on the pressure measured and the time elapsed, in a two dimensional matrix and/or for points on the body coordinate system. The risk indices may be presented as a map—step 714. The map displayed may provide an ongoing record of ongoing risk of a subject developing pressure related injuries which is in a form readily accessible to a caregiver.

Since an injury prevention system may be configured to detect pressure ulcers over the surface of a patient's body, the pixel coordinate based risk index function r(τ,x,y) described hereinabove:

$$r ( τ , x , y ) = {\sum }_{t = 1}^{τ} K ( t - τ ) * p ( P ( τ , x , y ) ) * {s}_{u} ( x , y , w , α )$$

may be of limited scope as it associates risk with points upon the supporting surface, such as a mattress or the like. Accordingly, it may be appropriate to describe the risk index function in the body coordinate system rather than a mattress coordinate system.

Moreover, by ignoring the body coordinates transform, under certain conditions such as where a subject's position moves relative to the pressure sensing assembly during measurement, the pixel coordinate based risk index function may generate inaccurate results. By calculating the risk index for locations in the body coordinate system, and recalibrating for each relative movement, such inaccuracies may be averted.

A body coordinate based risk index function may be defined, for example by the formula:

$$r ( τ , {x}_{u} , {y}_{u} ) = {\sum }_{t = 1}^{τ} K ( t - τ ) * p ( P ( τ , {x}_{u} , {y}_{u} ) ) * {s}_{u} ( {x}_{u} , {y}_{u} , w , α )$$

where each point on the body surface may be represented by a body coordinate vector (xᵤ,yᵤ).

Accordingly, a risk transform may be defined to measure the possibility of pressure injuries such as stress sores developing. Risk index values may measure the risk that a particular region of interest may develop a pressure injury. The size of each region of interest may be defined by the limit of the resolution of the data collected. Where data is collected by a pressure sensing assembly such as described herein, the smallest region of interest may be defined by the size of the pressure sensing elements, for example the intersections of the conducing strips in a pressure sensing mattress.

The risk transform may be used to generate solutions for the problem and may further develop more accurate formulas based on probabilistic theory. Accordingly, methods for describing the state of risk for a subject, such as visual methods for displaying the data or analytical methods for calculating a state of risk for a subject, may present monitored pressure data as a risk transform. This may enable a standardization of the analysis and the presented data. Moreover value standardization may enable the ready comparison between different methods.

The risk transform may be unit-less, having values referring to probabilities or pseudo-probabilities. For an initial calculation, the risk may be formulated as an approximate probability without necessarily preserving a full probabilistic formulation of the risk. Methods are presented which transform pressure values as measured, for example in millimeters of mercury, pascals, pounds, newtons or the like, to the risk transform in order to determine the risk of a region of interest developing a pressure injury such as a stress sore.

In one model, it may be assumed that occurrence of a pressure injury is a stochastic variable with a probability ‘r’ as defined by the risk index function. A probabilistic model may be simplified by assuming that all of the variables in the model, with the single exception of ‘occurrence of a pressure injury event’ (PSE) may be independent stochastic variables. Accordingly, using a Bayesian model, the risk index function r may be represented by:

$$r ( τ , {x}_{u} , {y}_{u} ) = P {r}_{τ , {x}_{u} , {y}_{u}} ( PSE \vert P ( τ , {x}_{u} , {y}_{u} ) , u , w , a , r ( τ - dt , {x}_{u} , {y}_{u} ) ) = \frac{P {r}_{τ , {x}_{u} , {y}_{u}} ( P S E ) P {r}_{τ , {x}_{u} , {y}_{u}} ( P ( τ , {x}_{u} , {y}_{u} ) \vert PSE ) P {r}_{τ , {x}_{u} , {y}_{u}} ( u \vert PSE ) {Pr}_{τ , {x}_{u} , {y}_{u}} ( w \vert PSE ) {Pr}_{τ , {x}_{u} , {y}_{u}} ( a \vert PSE ) P {r}_{τ , {x}_{u ,} {y}_{u}} ( r ( τ - dt , {x}_{u} , {y}_{u} ) \vert PSE )}{P {r}_{τ , {x}_{u} , {y}_{u}} ( P ( τ , {x}_{u} , {y}_{u} ) ) P {r}_{τ , {x}_{u} , {y}_{u}} ( u ) {Pr}_{τ , {x}_{u} , {y}_{u}} ( w ) P {r}_{τ , {x}_{u} , {y}_{u}} ( a ) P {r}_{τ , {x}_{u} , {y}_{u}} ( r ( τ - dt , {x}_{u} , {y}_{u} ) )}$$

Such a formula may be used to generate probabilistic estimations of the risk of each region of interest developing a pressure injury given current pressure conditions. Empirical data regarding the probability of pressure injury occurrence for various conditions may be gathered in preliminary data collection operations or accumulated over time. Such empirical data may be embedded in the formula in order to obtain the required risk index for each point on the surface of a body.

According to one algorithm, risk may be measured by recording a pressure distribution image of a subject, identifying the posture of the subject, mapping the pixels of the pressure sensing apparatus to a body coordinate system and calculating the risk of developing pressure injuries for each point on the body coordinates according to a formula such as the one outlined above.

The accumulated risk index may be presented to a caregiver as a visual display, for example on a body model, a rectangular array, a pressure distribution map or the like. It is particularly noted that a common risk index parameter may summarize the pressure risk values on the surface of a subject possibly facilitating the quantification and analysis of a subject's condition by a caregiver.

Various simplifications may be used to enable the assignation of risk index for monitored pressures. For example a non-linear relationship may be defined between pressure and risk or a pressure threshold may be established above which the accumulated pressure may be deemed high risk. Additionally or alternatively, a sigmoid weighting threshold function, W˜, may be used to adjust the pressure or any risk estimation by a multiplication between the values.

Accordingly, a single parameter measurement, the total risk R, may be calculated using the formula

$$R ( t = T ) = {\sum }_{x , y} {risk}_{t} ( x , y ) * {Wg}_{t} ( x , y )$$

Referring now to the flowchart of FIG. 9, a method is presented for determining the risk of a subject developing a pressure injury. The method includes monitoring pressure values for a set of pixels—step 902, for example, pressure may be measured using a set of pressure sensing elements corresponding with an area of overlap between a subject and a pressure sensitive sheet. Optionally, each of the pixels for which a pressure value has been monitored may be mapped to a body element—step 904.

An initial risk index may be set for each pixel or body element—step 906. After a certain duration, the time elapsed may be recorded—step 908 and a risk increment may be calculated for the pixel—step 910. The risk increment may be a function of the time elapsed and the pressure recorded during the elapsed time.

The risk increment may be added to the previous risk index to provide a new risk index—step 912. This risk index may be registered—step 914, for example by saving its value to a database of risk index values or the like.

Where appropriate, the risk index may be presented upon a visual display—step 916, perhaps using a color coded pressure risk map, a projection of pressure risk value representations onto a body model or the like. Such a display may provide a caregiver with an intuitive indication of risk of a subject developing pressure injuries and of possible preventative actions which may be taken to avoid such injuries developing.

As noted above, for various applications, it may be useful to identify body posture of the patient. Such identification may enable body features to be recognized or a body coordinate system to be mapped. It is noted that by recording a series of body postures adopted by a subject, it may be possible to determine other factors such as the risk of the subject falling from a bed or the like. Furthermore, knowing a subject's posture history may assist caring staff such as nurses or the like to choose a suitable new posture in which to reposition the subject when necessary.

Recumbent postures may be broadly classified by the orientation of the subject such that a posture where a subject is lying on her back may be termed a supine posture, a posture where a subject is lying on her front may be termed a prostrate posture, a posture where a subject is lying on her left side may be termed a left leaning posture and a posture where a subject is lying on her right side may be termed a right leaning posture.

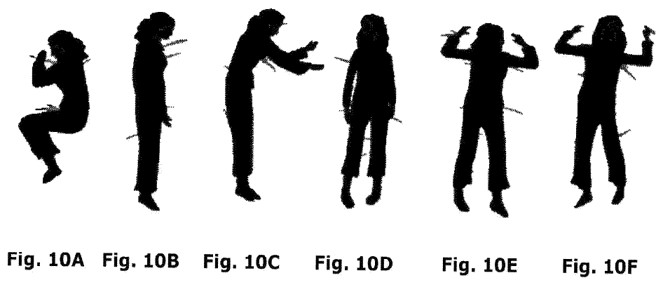

Referring now to FIGS. 10A-F, six body profiles are shown representing a selection of common postures adopted by subjects recumbent upon a horizontal surface. The postures shown illustrated some general posture classes adopted during sleep. FIG. 10A shows a right leaning posture known as ‘foetus’ which is adopted by about 41% of recorded sleepers, it will be appreciated that an equivalent left leaning ‘foetus’ posture may also be adopted. FIG. 10B shows a left leaning posture known as ‘log’ which is adopted by about 15% of recorded sleepers, it will be appreciated that an equivalent right leaning log posture may also be adopted. FIG. 10C shows a left leaning posture known as ‘yearner’, which is adopted by about 13% of recorded sleepers, it will be appreciated that an equivalent right leaning yearner posture may also be adopted. FIG. 10D shows a supine posture known as ‘soldier’, which is adopted by about 8% of recorded sleepers. FIG. 10E shows a prostrate posture known as ‘freefaller’ which is adopted by about 7% of recorded sleepers. FIG. 10F shows a supine posture known as ‘Starfish’ which is adopted by about 5% of recorded sleepers. It will be appreciated that further postures may be adopted particularly in hospital environments where subjects may have various injuries or ailments making adoption of common postures difficult or impossible.

It is noted that methods and systems of the disclosure may be able to identify such general posture classes. Furthermore each of the general posture classes listed above may include multiple variations. For example, a subject may lean to the right or the left, limbs may be shifted to various angles, and the head may be turned to right or left and the like. Systems and methods described herein may be utilized to distinguish between these variant postures and posture categories. By identifying postures, the position of the limbs may be identified and pixels may be mapped to a body coordinate system as required.

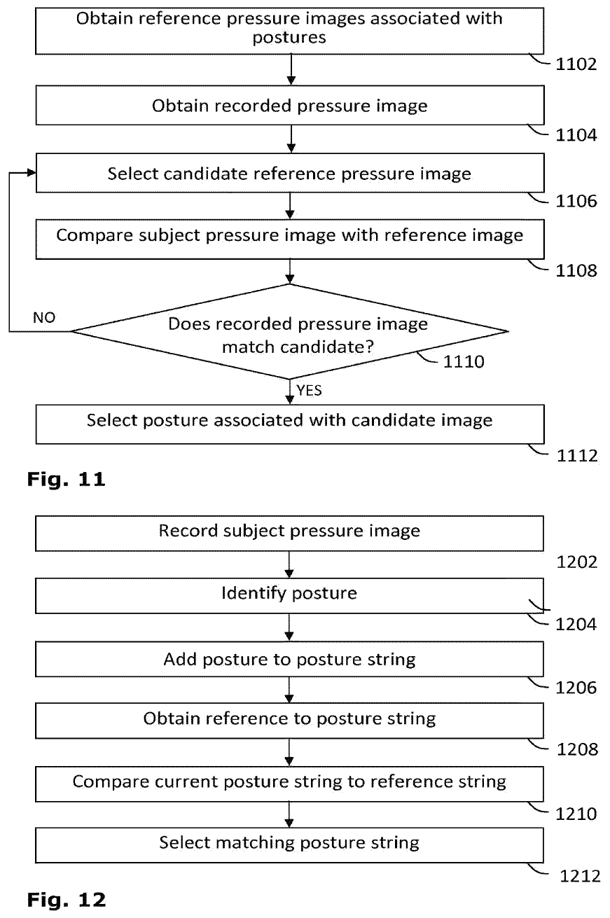

Referring now to the flowchart of FIG. 11 various selected actions are illustrated of a method for identifying a posture adopted by a recumbent subject. The method is executed by a processor associated with a pressure wound prevention system for example. The method may include obtaining a set of reference pressure images, each of the pressure images associated with a known posture—step 1102. It is noted that such a library may be collected by recording a sample of subjects adopting known postures and storing the pressure images or their associated pressure histograms in a posture library.

A pressure sensing assembly may be used to obtain a recorded pressure image of the subject—step 1104 for comparison with the reference pressure images in the posture library. A first candidate pressure image may be selected from the posture library 06 and compared with the recorded pressure image of the subject—step 1108.

If the candidate pressure image does not match the recorded pressure image—step 1110, a new candidate pressure image may be selected and compared to the recorded pressure image.

This may be repeated until the selected pressure image candidate matches the recorded pressure image. At this stage the known posture associated with the matching candidate pressure image may be selected—step 1112 and identified with the recorded pressure image.

As noted above, various comparison algorithms may be used to compare the recorded pressure image to the candidate images, such as particle component analysis, support vector machine, K-mean, two-dimensional fast Fourier analysis, earth movers distance and the like.

In some methods, the images may be compared indirectly by comparing the pressure distribution histograms of the recorded image and the candidate image. Indeed where appropriate only candidate histograms may be stored in the posture library. The earth mover distance (EMD) algorithm, for example may be used to compare between a first pressure distributions associated with a recorded posture and a second pressure distributions associated with candidate posture types stored in a database.

Optionally, the system may further record the duration during which the subject adopts each recorded posture.

A pressure distribution histogram may be obtained by creating a one dimensional array, or vector, of pertinent data relating to a pressure image feature. The histogram may serve as a signature of the pressure image feature and a comparison method may be used to provide a similarity rating between feature signatures.

It is noted that various methods may be used to generate pressure distribution signature vectors from pressure distribution images. For example, a signature vector of a maximum point distance feature may be obtained by: removing pixels having pressure values below a first threshold; identifying local maxima by selecting pixels whose pressure values are greater than or equal to all bordering pixels; clustering the local maxima into sets of a given size; obtaining a point average for each set of maxima, perhaps by calculating a spatial average therefor. Accordingly, an output vector may be generated arraying the distances between the local maxima average points.

Another method may be used for generating a pressure distribution signature vector comprising a histogram of pressure values. Optionally, the pressure value of each pixel may be arrayed into a histogram of total pressure values. Alternatively, or additionally, a partial pressure histogram may be generated by: calculating a spatial average for all values below a threshold value, the values being weighted for their positions; calculating the spatial averages; choosing a square of twice the standard deviation of data relative to the average position point; calculating a histogram of values out of this square.

Still another method may be used for generating a pressure distribution signature vector based upon the position of the monitoring pixels. The values of pixels may be selected where the pixel location is within a defined range.

A pressure sensing assembly may include a pressure sensitive pixels may include two sets of substantially orthogonally orientated conductive strips separated by an insulating layer of isolating material. The capacitive junctions formed at the intersections of two conductive strips may serve as the pressure sensors. Applying pressure to the sensor would compress the insulating layer, changing the distance between the conductive strips and thereby changing the capacitance of the capacitor.

Accordingly, an x-position histogram may be calculated by summing all the pressure values along each conducting strip parallel to the y-axis and arraying the total pressure values for each x-position. Optionally the x-position histogram values may be normalized by their sum. Similarly, a y-position histogram may be calculated by summing all the pressure values along each conducting strip parallel to the x-axis and arraying the total pressure values for each y-position. Optionally the y-position histogram values may be normalized by their sum.

Another method may be used to identify the outline of a body upon the pressure sensing apparatus. A background threshold may be determined. Pixels may be selected having pressure values below the background threshold which are also adjacent to at least one pixel having pressure values above the background threshold. The location of the selected pixels may be recorded, for example the row and column values, in a set of outline pixels. The adjacent points in the set of outline pixels may be joined to create a closed outline of the shape. A histogram may be obtained for the curvatures or torsion of the outline.

It will be appreciated that all the signatures described herein may be normalized to produce a normalized signature as required.

Comparison algorithms may use preliminary offline training during which certain patterns may be extracted from a training set of data. Patterns may be related to important, common, frequent or recurring patterns identified in the signatures. The extracted patterns may be used to match signatures with reference values.

As noted herein, various methods for comparison may be used. The Earth mover's algorithm (EMD) for example, may be used to compare histograms of similar sizes. Generally, the EMD may search for matching between bins of two histograms minimizing the multiplication of their values and distance. This may be refined by using several parameters to modulate the optimization methods to refer to several specific conditions.

Alternatively or additionally, Particle Component Analysis (PCA) may use a subset of an Eigen vectors transform to retrieve a principle component of the data having significant components to explain the variability in the data. A training set and an optimization process may be used to extract such principles. A subset of principles may be used, possibly online, for correlating against stored data perhaps using an inside multiplication. For example, the most significant component may be the average and may have the highest Eigen value. The training set could be used as Eigen vectors by simply calculating averages of different postures images. Then, their correlation with reference pressure images could be used to extract a score value indicating the similarity of the components to reference pressure images. In this manner, monitored pressure distribution images may be classified.

Referring now to the flowchart of FIG. 12, selected actions are presented of a method for identifying a sequence of postures sequentially adopted by a subject. The method may be useful in the generation of a posture string for example for use in predicting subject activity, in particular the risk of bed fall or bed exit. The method may include recording a subject pressure image—step 1202, possibly using a pressure sensing assembly as described herein. The posture may be identified—step 1204, for example as described above in relation to FIG. 11. The identified posture may be added to a posture string—step 1206, optionally together with labels indicating the duration that each posture is adopted.

A reference set of posture strings may be obtained—step 1208, for example from a posture string library stored in a database or the like. The current recorded posture string may then be compared to a reference posture string—step 1210. A matching reference posture string may then be selected—step 1212 indicating the activity of the subject.

A posture string may indicate a high risk of bed fall. For example, if a subject transitions sequentially from ‘right leaning log’ to ‘supine soldier’ to ‘left leaning log’ to ‘prostrate soldier’ to ‘right leaning log’ again, this may indicate that the subject is rolling to the left. Knowledge of such activity may be useful for the caring staff, who may be alerted to prevent the subject from falling from the bed or other supportive surface.

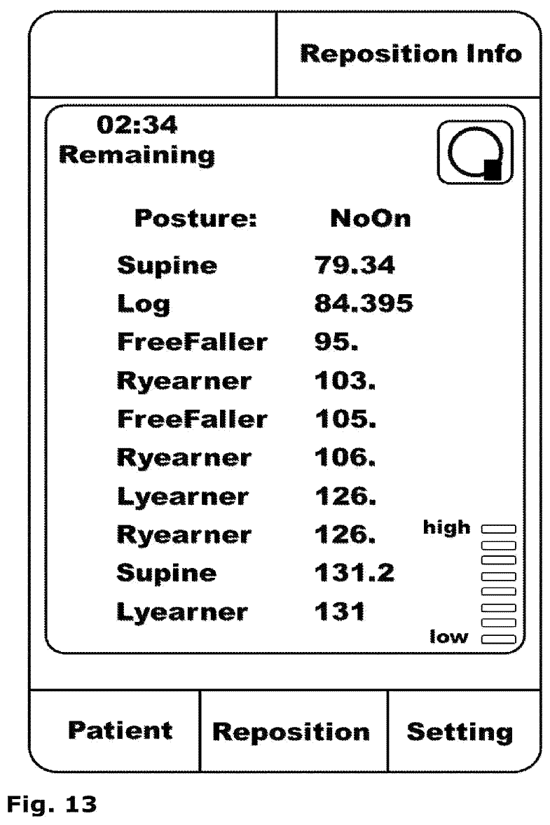

With reference now to FIG. 13 a possible screenshot is shown of a user interface. The screenshot indicates a list of postures together with the duration for which each posture was adopted. It is noted that such as list may provide indication for a caregiver to determine how best to reposition the subject. Indeed in some embodiments, a method may automatically calculate a preferred ‘next posture’ to be adopted by the subject.

Furthermore the posture string may indicate to a manager or supervisor whether the caregiver responsible for the subject was performing adequate steps to prevent the development of pressure injury. If, for example, a long duration is indicated for a single posture, this may indicate that the subject was not repositioned during that time. It is noted that such a system may provide a strong motivating factor for the caregiver.

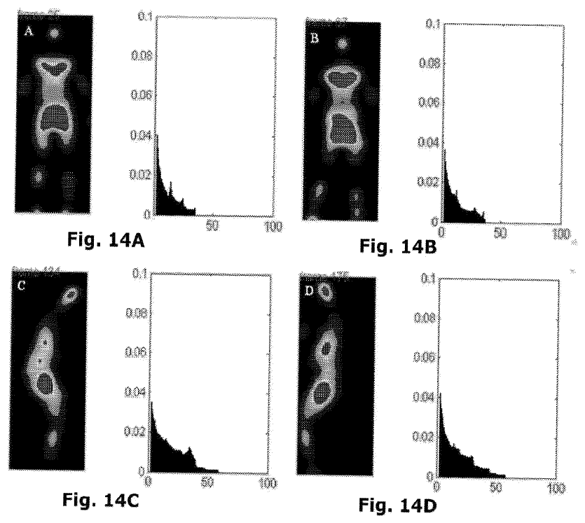

Reference is now made to FIGS. 14A-D showing four pressure distribution maps and associated pressure histograms recorded by a pressure sensing assembly for a subject adopting various recumbent postures. FIG. 14A shows a pressure map and associated pressure distribution histogram for a subject adopting a supine posture. FIG. 14B shows a pressure map and associated pressure distribution histogram for a subject adopting a prostrate posture. FIG. 14C shows a pressure map and associated pressure distribution histogram for a subject adopting a left leaning posture. FIG. 14D shows a pressure map and associated pressure distribution histogram for a subject adopting a right leaning posture.

It is noted that the pressure distribution histograms of each posture each have distinctive features which may be used to assist in the posture identification described hereinabove. It is further noted that smoothed and filtered histograms may produce more stable features for use in the comparison. Accordingly, exceptionally high values or other spikes may be discarded, the results may be normalized or the like.

Furthermore, it is noted that pressure distribution histograms may be used to examine the pressure map condition and to compare between different risk conditions. Histograms may be normalized variously, by number of pixels to achieve an absolute distribution risk/pressure maps, by number of nonzero pixel-values to achieve a body size relative distribution risk/pressure map or the like.

As noted above, for various applications, it may be useful to identify body posture of the patient. Such identification may enable body features to be recognized or a body coordinate system to be mapped. It is noted that by recording a series of body postures adopted by a subject, it may be possible to determine other factors such as the risk of the subject falling from a bed or the like. Furthermore, knowing a subjects posture history may assist caring staff such as nurses or the like to choose a suitable new posture in which to reposition the subject when necessary.

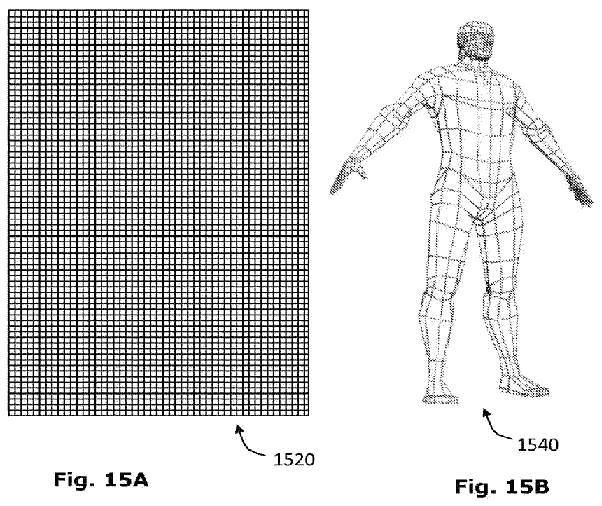

Referring to FIGS. 15A and 15B graphical illustrations are presented representing, respectively, a coordinate system of the pressure sensing assembly 1520 and a coordinate system of a subject body 1540. The pressure sensing elements of the pressure sensing assembly may be arranged as a two dimensional surface 1520, possibly as a rectangular arrangement or the like. The mapping of this two dimensional surface 1520 to a subject body coordinate system 1540 is a complex procedure.

The subject body is a three dimensional structure and the surface in contact with the pressure sensing elements is of two dimensions. However, the contact area between the subject body and the pressure sensing assembly may change with the movements of the subject, or movements of the pressure sensing assembly itself. Thus different pressure images and different associated postures may require different mappings between points on the body surface and sensing elements.

A pressure distribution image as recorded by the pressure sensing assembly may represent the pressure P exerted by the subject as measured by each of the pressure sensing elements. Each pressure sensing element may be situated at a known point which may be represented by a location vector in the coordinate system 1520 of the pressure sensing assembly. Accordingly, the recorded pressure value for the element may be associated with the location vector in the coordinate system 1520 of the mat. The location vector associated with each pressure value of the pressure distribution image coordinate system may be transformed to a mapped vector in a subject based coordinate system 1540.

The transformation from a location vector in the coordinate system of the mat 1520 to a mapped vector in the subject based coordinate system 1540 may require the identification of the current posture. This may allow landmark body points, to which a body coordinate system may be anchored, to be determined. Current posture may be identified, for example, using known algorithms such as particle component analysis, support vector machine, K-mean, two-dimensional fast Fourier analysis, earth movers distance and the like. In particular the earth mover distance (EMD) algorithm is a method to compare between two distributions, and which is commonly used in pattern recognition of visual signatures. The EMD algorithm may be readily applied, for example, to compare between a recorded posture and candidate posture types stored in a database. It is noted that the identification of particular body regions may have further application, for example in enabling a pressure wound prevention system to associate a particular pressure value with the relevant body region for more accurate calculation of a risk index function for that point.

In order to display any time dependent measurement which relies on body—local pressure values, it may be useful to track a body region of interest over time. Accordingly, it may be useful to detect such body regions of interest so that recorded pressure values and their changes over time may be associated therewith.

In general an algorithm may be used to receive an input of an array, possibly a rectangular array 1520, of pressure values from a pressure sensing assembly; and to return an output of pressure values associated with body coordinates 1540.

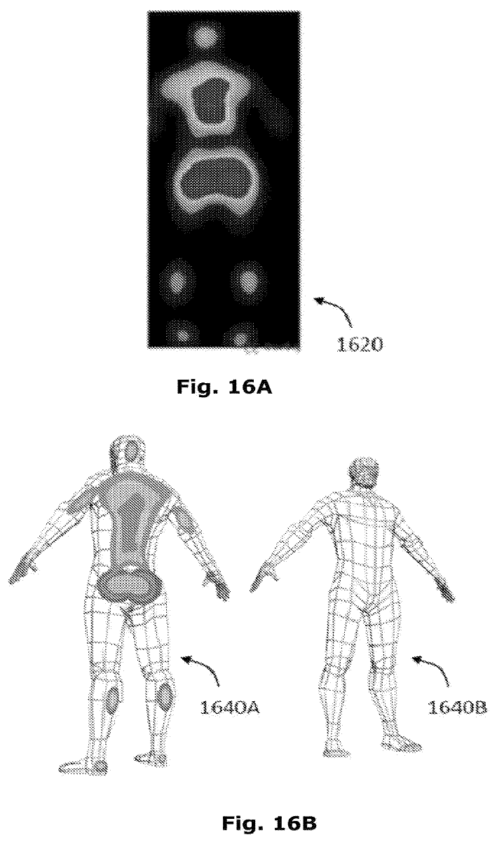

FIGS. 16A and 16B represent a possible pressure distribution image 1620 as recorded by a pressure sensing assembly and an associated body model 1640A, 1640B (referred to hereinafter collectively as 1640), respectively. The pressure distribution image 1620 may be collected, for example, when a subject is recumbent upon a surface in a supine posture, possibly identified as ‘soldier’ for example. The body model may have a back aspect 1640A and a front aspect 1640B.

A body model 1640 may be defined, for example, by identifying the posture of the subject from the pressure distribution image 1620 and identifying key body features from the identified posture. The body model 1640 may be used to calibrate the system by associating each pixel of the pressure distribution image 1620 corresponding to a point of contact between the pressure sensing assembly and the subject with a region on the surface of the subject's body.

Once the pixels are mapped to the body surface, the corresponding pressure records or calculations may be associated with the relevant body regions and the pressure distribution image 1620 may be reconstructed by projecting the pressure values of each pixel onto the body model 1640. It will be appreciated that as the subject position changes or a new posture is identified, the mapping may be recalibrated. Accordingly, where appropriate, pressure records may remain associated with the same body regions over time even when data is collected from different pressure sensing elements.

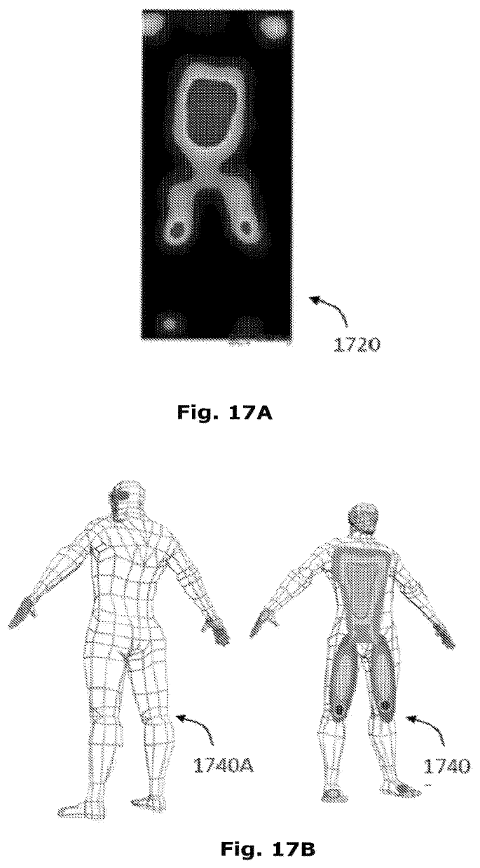

Referring now to FIGS. 17A and 17B another possible pressure distribution image 1720 and its associated body model 1740A, 1740B (referred to hereinafter collectively as 1740) are represented for a subject recumbent in a prostrate posture, such as ‘free fall’ for example. The pressure distribution image 1720 may be recorded by a pressure sensing assembly.

Comparing FIGS. 16A and 16B to FIGS. 17A and 17B, it is apparent that different areas of the body model are highlighted, indicating that different regions on the surface of a body of a subject are under pressure in each posture. In particular, the supine posture of FIGS. 16A and 16B has most of the pressure exerted upon the rear of the subject, with highest pressure around the buttocks and upper back whereas in the prostrate posture most of the pressure is exerted upon the front of the subject, with highest pressure around the chest and knees. It is noted however that certain regions, such as the left hand side of the head, for example, may be pressured in both postures.

Body tracking across a plurality of postures may be managed using a variety of methods. A first method for tracking body regions may use a two dimensional pressure distribution image with defined regions to describe a body model and a Gaussian model to estimate the projection of pressure data from the pressure sensing assembly into those images.

Accordingly, the body model may be defined by a selection of postures such as Supine, Left Yearner, Right Yearner, or the like. The body model may specify two dimensional images of a recumbent subject in each of the associated postures.

The system may be calibrated for each posture image associated with a pressure image from the pressure sensing assembly. Transformations may be performed between the pressure distribution images describing the postures and their model image. Optionally, this could be achieved by a manual procedure in which regions of the body may be associated with associated regions in the model. For example, the arms in the supine posture, say, of the body model may be associated with the pixels associated with the arms from the pressure distribution image.

The association between pixels and body regions may be indicated, for example, by coloring, perhaps using painting software, a pressure distribution image and a body model such that matched regions share a common color. Each region of pixels may then be used to reconstruct a three dimensional Gaussian fit. The Gaussian pixels associated with a particular body region and the corresponding Gaussian pixels associated with the pressure distribution image may then be matched using a transformation, for example related to calculations involving their standard deviations and mean values. Such a transformation may be used to back project pressure values recorded at pixels of the pressure distribution image onto the body model.

The body model pressure distribution image may be reconstructed for a plurality of subject postures by identifying each posture, perhaps using a posture detection algorithm. An isodata method, such as a Gaussian mixture model or vector quantization model may use a maximal likelihood method to cluster pixels into regions. The clustering may use three dimensional Gaussian pixel calculations performed during the posture identification. Each cluster of pixels may be used to calculate a Gaussian fit. Accordingly, a three dimensional Gaussian fit may be generated for each cluster, and any pixel from a target pressure distribution image may be projected into an appropriate pixel in the model image by transforming the target image Gaussian fit to the calibration pressure distribution image and then from the calibration pressure image to the model pressure image.

A biomechanical solution for the tracking problem may be used to take into account the most important biomechanics properties of the human body and to apply them to the model used in the calibration process. One advantage of such a solution would be that biomechanics may introduce certain time dependent constraints to possible values. This may reduce noise and processing power during limb detection.

Additionally, or alternatively, the output of a biomechanical algorithm may include measurements of the values for the degrees of freedom in the biomechanical model. For example, where the body model is represented by a set of straight nonflexible sections connected by pivots with limited degrees of freedom, perhaps defined by limiting angles or the like, the output of the algorithm should include the values of the limiting angles of the pivots, the locations of each section and the like. The reverse engineering problem involving such angles and body section positions may be used to determine the local pressure values.

Accordingly an error function may be defined which represents the difference between the target image to be projected onto the body model and the estimated projection of the biomechanical model onto the pressure sensing assembly. By minimizing such an error function, the biomechanical model may be used to closely approximate the projected image.

Various methods may be used to minimize the error function. By way of a nonlimiting example, one minimization method may include: determining the posture by using a posture detection algorithm; selecting a random initial set of values for body parameters such as angles, body part locations and the like; generating a calibration image for the identified posture, optionally using the algorithm appropriate posture image and adjusting body parameters, for example by rotating or aligning each body section, by a set of shift values determined from the initial set of values for body parameters; clustering the target image, for example using an isodata method such as described herein; calculating an updated set of values for the body parameters possibly by using a Gaussian estimation such as described herein; and repeating the process until a minimum value is obtained for the error function.

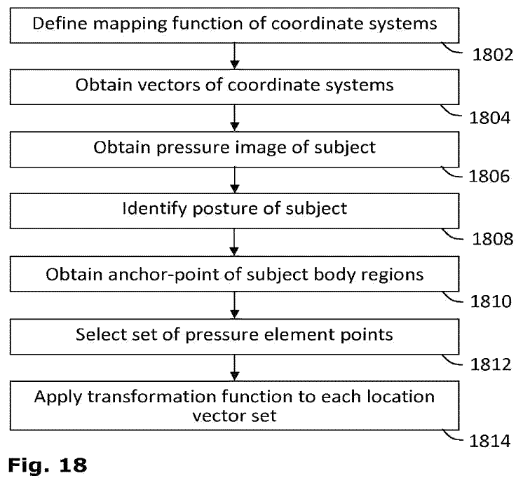

Referring now to the flowchart of FIG. 18 various selected actions are illustrated for a possible method for mapping the coordinate system of a subject adopting various recumbent postures. The method may be executed by a processor associated with a pressure wound prevention system for example. The mapping may involve associating a set of location vectors for pressure sensing elements of the pressure sensing assembly to a set of mapped location vectors in a body-based coordinate system.

A subject coordinate system mapping function is defined—step (1802). The function of the coordinate system mapping may include obtaining vectors specifying the coordinate systems, obtaining a pressure image of the subject and identifying the current posture, obtaining body regions and using the appropriate transformation function as described hereinabove.

The method may include obtaining a vector for the subject based coordinate system and for the coordinate system of the pressure sensing assembly surface of the sensing elements-step (1804). The pressure sensing assembly may be used to obtain a recorded pressure image of the subject—step (1806). The posture of the recumbent subject may be identified—step (1808), for example by comparison with the reference pressure images in a posture library. It is noted that such a library of postures may be collected by recording a sample of subjects adopting known postures and storing the pressure images or their associated pressure histograms in a posture library.

Anchor-point subject body regions may be obtained—step (1810). The anchor-point body regions may be obtained variously as part of the posture identification process, as part of the transformation process, as a default set, obtained from some repository, entered by a user or the like. Optionally, body regions may be defined in broad terms such as ‘upper’, ‘lower’, and ‘left’ or ‘right’ or specific limbs like ‘leg’, ‘arm’, ‘head’ and more.

A set of pressure elements may be selected from the pressure sensing elements—step (1812), for example the set of pressure sensing elements corresponding to the contact area may be selected. Each element of the selected set of pressure sensing elements may be transformed to the coordinate system of the body—step (1814), possibly using a function such as noted hereinabove.

The process may be repeated until all selected pressure sensing element points are transformed into the subject based coordinate system.

Additionally, aspects of the present disclosure relate to improving the monitoring of the pressure image indicating the patient's pressure distribution by reducing the noise signal from several sources.

A pressure wound prevention system may be used to monitor pressure distribution between a subject and a surface and to warn and/or alert a caregiver to potential risk of the subject developing pressure wounds. Using such a pressure wound prevention system may, therefore, enable the caregiver to take preventative action such as turning or otherwise repositioning the subject before pressure wounds develop.

As such, the pressure wound prevention system may need to analyse the movements of a subject on the pressure mat, identify the posture of the subject, identify the position of limbs, monitor pressure exerted upon the subject, characterize the pressure image of the subject and the like to provide appropriate care such as repositioning of the subject by the caregiver.

A pressure distribution image may be subject to various types of noise, such as interference signals which may add spurious data from various sources to meaningful data measurements. The display of the pressure distribution map may be enhanced by reducing the levels of noise sources which may appear while reading pressure mat measurements. In particular, methods may be used to reduce random noise using, for example, a motion dependent smoothing mechanism as well as methods for detecting and removing noise patterns in the background.

The motion dependent smoothing mechanism may assume measured pressure to be the sum of the actual pressure and an added noise component, which has a random value distributed symmetrically around a zero mean. Such a distributed noise component may be eliminated by averaging values recorded for each sensor or pixel since the last significant change in the pressure map.

A noise pattern background eliminator may detect background patterns, such as patterns detectable by eye, which may be eliminated using spatial convolution. More complex noise patterns, which may not be detectable by eye, may be eliminated using a more complex Fast Fourier Transform (FFT) analysis. Noise reduction may be achieved using a premeasured transform function, for example, derived from an analysis of signal and noise components.

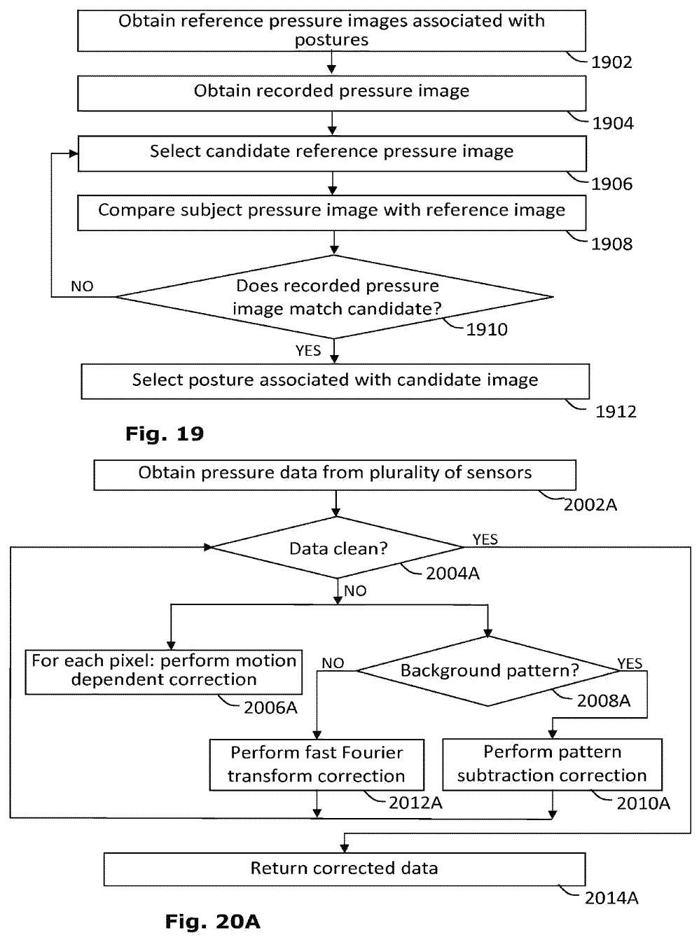

Referring now to the flowchart of FIG. 19, various selected actions are illustrated of a method for identifying a posture adopted by a recumbent subject. The method may be executed by a processor associated with a pressure wound prevention system, for example. The method may include obtaining a set of reference pressure images, each of the pressure images associated with a known posture—step 1902. It is noted that such a library may be collected by recording a sample of subjects adopting known postures and storing the pressure images or their associated pressure histograms in a posture library.

A pressure sensing assembly may be used to obtain a recorded pressure image of the subject—step 1904, for comparison with the reference pressure images in the posture library. A first candidate pressure image may be selected from the posture library—step 1906 and compared with the recorded pressure image of the subject—step 1908.

If the candidate pressure image does not match the recorded pressure image—step 1910, a new candidate pressure image may be selected and compared to the recorded pressure image.

This may be repeated until the selected candidate pressure image matches the recorded pressure image. At this stage, the known posture associated with the matching candidate pressure image may be selected—step 1912, and identified with the recorded pressure image.

As noted above, various comparison algorithms may be used to compare the recorded pressure image to the candidate images, such as particle component analysis, support vector machine, K-mean, two-dimensional fast Fourier analysis, earth movers distance and the like.

In some methods, the images may be compared indirectly by comparing the pressure distribution histograms of the recorded image and the candidate image. Indeed where appropriate only candidate histograms may be stored in the posture library. The earth movers distance (EMD) algorithm, for example, may be used compare between two pressure distributions having recorded posture and candidate posture types stored in a database.

Optionally, the system may further record the duration during which the subject adopts each recorded posture.

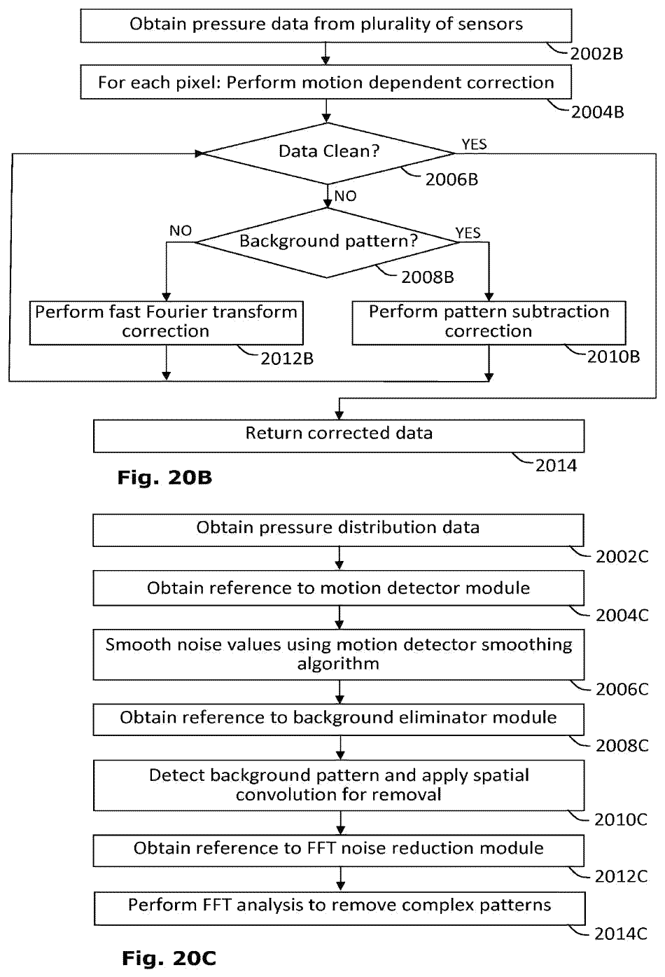

Referring now to the flowcharts of FIGS. 20A-C, various selected actions are indicated of a method for analyzing and reducing possible noise patterns from different sources, and may possibly be activated in different configurations. The method may be used and executed by a processor associated with. As indicated hereinabove, the pressure distribution image is subject to a

As indicated hereinabove, the pressure distribution image is subject to a variety of noise interfering with the accuracy of pressure distribution measurements. Elimination of at least some of the noise may be effected through the application of at least one algorithm, such as random noise smoothing, background noise pattern elimination using a convolution between the spatial map and the measured pressure, and analysis using Fast Fourier Transform (FFT) noise reduction for complex, nonlinear noise patterns. h a pressure wound prevention system, for example.

The motion dependent smoothing mechanism is directed towards eliminating random noise. Random noise is considered to comprise a noise component to the measurement of each sensor, which is distributed symmetrically around a zero mean. According to Chebyishev's inequality, as the number of samples increases, the mean value becomes a better estimator for the actual measurement as it excludes symmetric noise components. Thus, by collecting more measurement samples, the average measurement calculation may produce a more accurate indication of pressure measurement.

Furthermore, the suggested mechanism assumes that there exists a measurable movement-dependent minimal threshold. A value below the threshold indicates no movement on top of the pressure mat. Accordingly, any variation about the mean of the pressure recorded is associated with a noise source.

Based on the hereinabove assumptions, the mechanism for providing a pressure distribution map representing data gathered from a pressure detection apparatus comprising a plurality of sensors may be configured to monitor pressure exerted by a subject on a surface. Pressure may be recorded at each sensor element, at time intervals to determine the output pressure. The output pressure may be calculated for each pixel or may be the average value of all values measured for example. This output pressure value may be returned or recorded for an optional usage, such as to form a pressure distribution image map of the subject, for example.

Various methods may be used to obtain a motion dependent grade. In general, a motion grading function may be a comparative function of pressure map sequence over time. For example, such a grade could be calculated for a given time t by summing all the absolute differences between two adjacent frames, acquired at times t and t-l, corresponding to the pixel's pressure values. Other methods may consider longer sequences of pressure map frames and/or other formulations to achieve such a calculation. Additionally or alternatively the sum of squares of the differences could be used. It is noted that in some cases, an average may be more efficient when computing the sum of a large number of difference values because by dividing the sum by the total number of differences, the resulting average differences are smaller. In general this could be denoted by:

$${m}_{t} = Grade ( T ) = Mean ( Sum ( K ( t ) * P x y ( t ) , t = 0 , t = T ) , x , y )$$

where Pxy denotes the pressure of pixel xy at time t, and K(t) is a kernel function giving the signed weight of the pressure value in time t.

Using the above formulation in the above described case of absolute differences we may use this formulation:

$${m}_{t} = Grade ( T ) = Mean ( abs ( Pxy ( t ) - P x y ( t ) ) , t = 0 , t = T ) , x , y )$$

$$where K ( t = T ) = 1 , or else K = 0 ;$$

Movement of the subject is monitored to determine if actual movement takes place; when the motion indicator value is greater than a threshold value, then movement is considered to have happened. At this point, the calculation of the average pressure output is restarted.

The below describes the mechanism of average pressure calculation, based on movement indications:

$${T}_{t} = \begin{matrix}\{ {T}_{t - 1} & {m}_{t} < {M}_{th} \\ \{ t & {m}_{t} \geq {M}_{th}\end{matrix}$$

$${y}_{t} ( x , y )$$

$$\begin{matrix}= \{ {f}_{t} ( x , y ) & if {T}_{t} = t\end{matrix}$$

$$\{ Sum ( {f}_{i} ( x , y ) , i = {T}_{t} , i = t ) / ( t - {T}_{t} ) else$$

where,
fₜ (x,y), is a matrix indicating the pressure measurements on top of pixel x, y at time t.
mₜ, is a measured movement on the pressure mat at time t.
Mₜₕ, is a threshold motion indicator, where movement is indicated if mₜ>Mₜₕ.
yₜ (x,y), is a matrix of a noise-reduced measurement of pressure at pixel x, y at time t.

The elimination of background noise patterns may be possible as it is partially visual and could easily be distinguished from true signals. These noise sources could be defined as background static noise.

The method assumes that for each noise pattern there is a well-defined spatial map denoting the spatial pattern of pressure reduction or amplification relative to a referenced (X, Y) pixel. This map could be denoted as a spatial map n_{xy} (X, Y), where x, y defines the referenced pixel and n_{xy} (X, Y) denotes the reduction or enhancement at pixel X+x, Y+y. The corrected value at pixel (X, Y) may be extracted by using a convolution between the map and the measured pressure. This convolution may compensate for the estimated interference denoted by the map and may also rely on cross correlation between adjacent pixels. Additionally, this convolution may mask the pixels which are not relevant for the calculation.

$$P ( x , y ) = sum ( f ( x , y ) * {C}_{x y} ( x , y , {n}_{x y} ) ) on all X + x , Y + y surrounding X , Y$$

where Cxy is the correcting convolution which considers n_{xy} (X, Y) and the cross correlations.

Additionally, Fast Fourier Transform (FFT) noise analysis may be applied for further reduction of a more complex pattern nature of the noise.

It may also be possible that some noise sources are more complex than could be detected visually. Except for random noise sources or constant spatial known patterns, which appear in the measurement as a linear function of the actual value, there may be non-linear deviations that are expressed as a function of the input. Those complex patterns could be determined in the noise samples and distinguished from the signal after extracting their properties using appropriate analysis.

A Fast Fourier Analysis, for example could be used for such a purpose (Weiner filtering) and point to noise-specific frequency components/patterns which do not exist in the signal. Once a noise component or pattern has been identified, a transformation method may be used to eliminate those components or patterns.

The method as indicated in the flowchart of FIG. 20A for analyzing and reducing noise may include gathering pressure distribution data—step 2002A, for example, by using a possible pressure sensing assembly, and testing the data quality of pressure measurements—step 2004A. If noise values are associated with the measurement, at least one of a plurality of possible noise reduction algorithms may be applied to the collected pressure data to remove various sources of noise.

Optionally, for each pixel of the pressure mat, a motion dependent correction may be performed—step 2006A, to reduce random noise components of the pressure distribution image having a symmetric distribution around zero. Additionally or alternatively, background patterns may be examined—step 2008A, if such noise patterns are detected, they may be reduced by performing a pattern subtraction correction—step 2010A.

Optionally again, further analysis may be enabled for detecting more complex noise nonlinear patterns, correcting these complex patterns using Fast Fourier Transform (FFT) noise reduction—step 2012A.

Additionally or alternatively, the corrected data may be tested if noise values are still associated with the measurement. If no further noise reduction is required, the corrected data may be returned—step 2014A.

The method as indicated in flowchart of FIG. 20B is another possible flow configuration for analyzing and reducing noise and may include gathering or providing pressure distribution data—step 2002B.

Optionally, for each pixel of the pressure mat, a motion dependent correction may be performed—step 2004B, in order to reduce random noise component of the pressure distribution image. Following the motion-dependent correction, the pressure data is tested for the presence of further noise—step 2006B. If noise is detected, testing for the presence background patterns may be applied—step 2008B. If a background pattern is detected, a pattern subtraction correction is performed—step 2010B. If a background pattern is not detected, then a Fast Fourier Transform correction is performed—step 2012B.

Additionally or alternatively, the corrected data may be tested for still existing noise levels. If no further noise reduction needs to be applied, the corrected data may be returned—step 2014B.

The method as indicated in flowchart of FIG. 20C is another possible flow configuration for analyzing and reducing noise. The method may include gathering or obtaining pressure distribution data—step 2002C.

At least one of a plurality of possible noise reduction algorithms may be applied to the collected pressure data to remove various sources of noise

Additionally or alternatively, the method may include a motion dependent detection module—step 2004C. Accordingly, motion dependent smoothing may be used to reduce random noise components of the pressure distribution image having a symmetric distribution around zero—step 2006C.

Additionally or alternatively, the method may include a background eliminator module—step 2008C. Accordingly, noise patterns may be detected in the background and may be reduced by applying a spatial convolution based algorithm—step 2010C.

Additionally or alternatively, the method may include a Fast Fourier Transform (FFT) noise reduction module—step 2012C. Accordingly, further analysis may be enabled for detecting more complex, nonlinear noise patterns. Such complex patterns may be analyzed, for example, in the noise samples and distinguished from the signal using Fast Fourier Transform corrections—step 2014C.

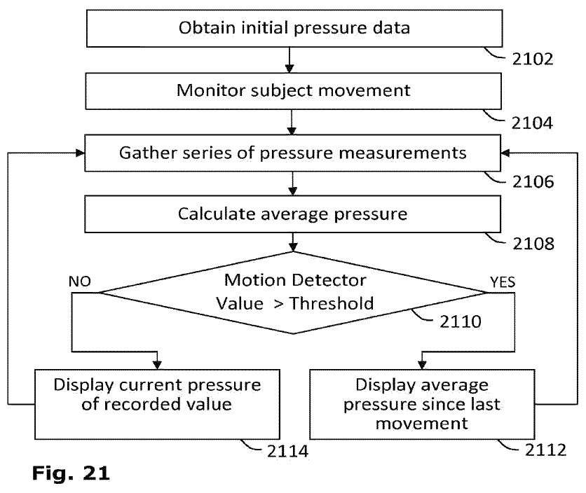

Referring now to the flowchart of FIG. 21, various selected actions are indicated of a method for reducing the possible random noise component of the subject's pressure distribution image. The method may be used and executed by a processor associated with an injury prevention system, for example.

The method may include: obtaining an initial pressure data—step 2102, using a possible pressure sensing assembly, for example. A motion detector monitors subjects' movement—step 2104 and a series of pressure measurements are gathered by the sensing elements—step 2106. The running average of the series of pressure measurements is calculated—step 2108. The motion detector may analyze actual body movements, possibly comparing a motion detector value (MDV) to a threshold value—step 2110.

Optionally, if the motion indicator value is greater than a threshold value, the average pressure value may be returned or recorded for an optional usage, such as to form a pressure distribution map, for example—step 2112 and current average value is set to the last measurement. If no motion is indicated, the actual pressure measurement may be returned or recorded for an optional use. Such use may be to form a pressure distribution map, for example—step 2114.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of terms such as computing unit, network, display, memory, server and the like are intended to include all such new technologies, a priori.

As used herein the term “about” refers to at least+10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

The scope of the disclosed subject matter is defined by the appended claims and includes both combinations and sub combinations of the various features described hereinabove as well as variations and modifications thereof, which would occur to persons skilled in the art upon reading the foregoing description.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. An injury prevention system comprising:
a plurality of pressure sensing assemblies configured to measure pressure exerted upon a plurality of subjects over time, each of the pressure sensing assemblies in the plurality of pressure sensing assemblies includes a hardware controller and a pressure sensor array; and
a management system including a plurality of system control units and a remote unit, the remote unit is remote from the plurality of system control units, each of the hardware controllers configured to transfer output signals from each of the pressure sensor arrays to one of the system control units, the remote unit is configured to communicate with the plurality of system control units to monitor the plurality of subjects for injury prevention.

2. The injury prevention system of claim 1, wherein the plurality of system control units are a plurality of bedside system control units.

3. The injury prevention system of claim 2, wherein the remote unit is located at a nurse's station.

4. The injury prevention system of claim 1, wherein the remote unit is configured to record data associated with the measurements of pressure exerted upon the plurality of subjects over time in a data storage unit for subsequent retrieval.

5. The injury prevention system of claim 1, wherein the remote unit is configured to alert a caregiver to a potential risk of one or more of the plurality of subjects developing an injury.

6. The injury prevention system of claim 1, wherein the remote unit is configured to an enable a user to configure settings.

7. The injury prevention system of claim 1, wherein the remote unit is configured to remotely monitor the output of the plurality of pressure sensing assemblies.

8. The injury prevention system of claim 1, wherein the remote unit and the plurality of system control units are configured for two-way communication.

9. The injury prevention system of claim 1, wherein each of the pressure sensing arrays is placed between a mattress and a subject from the plurality of subjects.

10. The injury prevention system of claim 1, wherein one or more of the pressure sensing arrays are pressure sensitive pads.

11. A pressure sensing system comprising:
a pressure sensing assembly comprising pressure sensing elements situated at known points represented by a location vector in a first coordinate system of the pressure sensing assembly; and
a non-transitory computer-readable medium having computer-readable instructions stored thereon that are configured to be executed by a processor to perform the following functions:
recording pressure values representing pressure exerted by a subject on the pressure sensing elements;
associating the recorded pressure values with the location vector of the coordinate system; and
transforming the associated location vector into a mapped vector in a second coordinate system to obtain a pressure distribution image.

12. The pressure sensing system of claim 11, wherein the first coordinate system is a two-dimensional coordinate system.

13. The pressure sensing system of claim 11, wherein the second coordinate system is a three-dimensional coordinate system.

14. The pressure sensing system of claim 11, wherein the second coordinate system is a body coordinate system.

15. The pressure sensing system of claim 14, wherein the computer-readable instructions are configured to be executed by the processor to further perform the following functions: anchoring the body coordinate system to one or more landmark body points.

16. The pressure sensing system of claim 11, wherein the known points are pixels.

17. The pressure sensing system of claim 11, wherein the computer-readable instructions are configured to be executed by the processor to further perform the following functions: associating the recorded pressure values with one or more body regions.

18. The pressure sensing system of claim 17, wherein the one or more body regions include upper or lower and left or right.

19. The pressure sensing system of claim 18, wherein the one or more body regions include upper or lower.

20. The pressure sensing system of claim 18, wherein the one or more body regions include left or right.
